(19) United States
(12) Patent Application Publication
(10) Pub. No.: US 2026/0284321 A1
(43) Pub. Date: Sep. 24, 2026

(54) A DRUG INJECTION DEVICE

(71) Applicant: NEMERA SZCZECIN SPÓLKA Z OGRANICZONA ODPOWIEDZIALNOSCIA., Szczecin (PL)

(72) Inventors: Mateusz WILCZEK, Szczecin (PL); Arkadiusz Lukasz WOITAS, Szczecin (PL); Gwenael JAILLET, Oullins (FR); Guillaume ALBRAND, Brignais (FR)

(21) Appl. No.: 19/469,554

(22) PCT Filed: Mar. 27, 2024
(86) PCT No.: PCT/IB2024/052943

§ 371 Date: Sep. 26, 2025

(30) Foreign Application Priority Data
Mar. 28, 2023 (PL) P444242

(51) Int. Cl. A61M 5/315

(52) U.S. Cl. CPC A61M 5/31526; A61M 5/31501; A61M 5/31536; A61M 5/31541; A61M 5/31551; A61M 5/31585; A61M 5/31593; A61M 2202/0007

(57) ABSTRACT

A drug injection device comprising a housing (1) comprising an internal thread (1.2), a push button (12) at its proximal end and, a driving assembly mounted in the housing, the driving assembly comprising: (i) an axially movable and rotatable dial sleeve (8) comprising an external thread (8.3) cooperating with the internal thread (1.2) of the housing (1), (ii) an axially movable and rotatable coupling element (10) within the dial sleeve (8), (iii) an axially immovable and rotatable driving sleeve (7) within the coupling element (10) and rotationally coupled therewith, the driving sleeve (7) comprising at least one radially extending ratchet arm (7.6) at its distal end (7.5), (iv) an axially movable and rotatable piston rod (6) comprising an external thread (6.1), the piston rod (6) being rotationally coupled with the driving sleeve (7), (v) a ratchet wheel (5) fixed in the housing (1) and comprising a series of radial teeth (5.4) cooperating with said at least one ratchet arm (7.6) for blocking rotation of the driving sleeve (7) in one direction and allowing rotation of the driving sleeve (7) in the opposite direction, the ratchet wheel (5) further comprising an opening (5.2) having an internal thread (5.3) cooperating with the external thread (6.1) of the piston rod (6), wherein the dial sleeve (8) comprises a proximal enlarged portion (8.2) comprising a first set of teeth (8.6) and a second set of teeth (8.8) located distally from the first teeth (8.6), the coupling element (10) comprises at least one axially extending structure (10.6), the structure (10.6) comprising a protrusion (10.8) engaging the first set of teeth (8.6) of the enlarged portion (8.2), the protrusion (10.8) passing over first set of teeth (8.6) into subsequent positions during dose setting and dose correcting, the coupling element (10) comprises the coupling teeth (10.9), the coupling element (10) being axially movable between a dose setting/correcting position in which the coupling teeth (10.9) are decoupled from said second set of teeth (8.8), and a dose delivery position in which the coupling teeth (10.9) are coupled with said second set of teeth (8.8).

The invention relates to drug delivery devices for subcutaneous injection. In particular, the invention relates to drug delivery devices for multiple injections of adjustable volume doses of a medicine.

The pen-injectors are the drug delivery devices for performing subcutaneous injections, preferably by a patient. They usually have a longitudinal shape facilitating better grasping of the device by the patient's hand. A medication to be injected is contained in a cartridge being usually a glass barrel closed by a septum from one side and by a rubber piston on the second side. A needle for connecting the cartridge interior with the subcutaneous tissue is attached at one end of the pen-injector so that it may pierce the septum and provide a fluidpath between the cartridge and subcutaneous tissue. The opposite end of the pen-injector is usually provided with activating means, i.e. the means for setting and/or injecting the dose. The activating means may be provided also in other locations, for example at the lateral surface of the housing.

Various types of pen-injectors include the disposable devices to be discarded once the cartridge is empty and the reusable devices in which the cartridge may be exchanged. A force required to push the piston and expel the drug may be provided a user. Another possibility is that the energy is provided by resilient means, for example a spring, which may be strained by a user during dose setting or preloaded during manufacture or assembly of the pen-injector. The pen-injectors may be designed so that the volume of the dose is fixed or adjustable by the user.

WO99/38554 discloses an injection syringe for apportioning set doses of a medicine from a cartridge. The dose is set by rotation of a scale drum, which is threadedly engaged with a housing. The injection is driven manually by a force provided by a user when an injection button is pressed. During the injection, the scale drum rotates causing rotation of a bushing and a driver tube, the rotation being further imparted to a piston rod. Between the driver tube and the housing, the injection syringe comprises a pawl mechanism allowing rotation of the driver tube only in one direction.

Another drive mechanism used in the pen-type injectors of same type is disclosed in WO2004/078239. To set a dose, a dose dial grip is rotated and a drive sleeve, a clicker, a clutch and a dial sleeve are rotated as well due to engagement between the clicker and the clutch. In order to inject the set dose, a user presses a button and the clutch is displaced axially and disengages from the dose dial sleeve. However, the clutch is still keyed to the drive sleeve. Hence the dial grip along with the dial sleeve are rotated back being guided helically, but the clutch and the drive sleeve move only axially. In this way the drive sleeve rotates a piston rod due to a threaded connection between these elements. The piston rod is simultaneously moved axially due to a second thread connection between the piston rod and an insert fixed in the housing.

The aim of the present disclosure is to provide an improved drug injection device which allows for easy and precise dose setting and correcting. In addition, any possible leakage of the drug during setting and correcting a dose is prevented. Further, a user is provided with audible feedback about the set dose and the injection by click sounds.

The drug injection device according to the present disclosure comprises a housing comprising an internal thread, a push button at the proximal end of the housing and a driving assembly mounted in the housing, the driving assembly comprising:
an axially movable and rotatable dial sleeve comprising an external thread cooperating with the internal thread of the housing,
an axially movable and rotatable coupling element within the dial sleeve,
an axially immovable and rotatable driving sleeve within the coupling element and rotationally coupled therewith, the driving sleeve comprising at least one radially extending ratchet arm at its distal end,
an axially movable and rotatable piston rod comprising an external thread, the piston rod being rotationally coupled with the driving sleeve,
a ratchet wheel fixed in the housing and comprising a series of radial teeth cooperating with said at least one ratchet arm for blocking rotation of the driving sleeve in one direction and allowing rotation of the driving sleeve in the opposite direction, the ratchet wheel further comprising an opening having an internal thread cooperating with the external thread of the piston rod.

The drug injection device according to the disclosure is characterized in that:
the dial sleeve comprises a proximal enlarged portion comprising a first set of teeth and a second set of teeth located distally from the first teeth;
the coupling element comprises at least one axially extending structure comprising a protrusion engaging the first set of teeth of the enlarged portion, the protrusion passing over the first set of teeth into subsequent positions during dose setting and dose correcting;
the coupling element comprises coupling teeth and is axially movable between a dose setting/correcting position in which the coupling teeth are decoupled from said second set of teeth of the dial sleeve, and a dose delivery position in which the coupling teeth are coupled with said second set of teeth of the dial sleeve.

The coupling element may be axially movable between the dose setting/correcting position and the dose delivery position by means of the push button.

The coupling element may be rotationally engaged with the push button by intermediary of a connector.

The coupling element and the connector may rotate with respect to the push button. The connector may comprise a ledge and the coupling element may comprise an internal shelf, the ledge being adapted to abut the internal shelf.

The connector may comprise at least two protrusions on its ledge and the coupling element may comprise openings on its enlarged part wall, the at least two protrusions being designed for engagement with said openings.

The push button may comprise a cylindrical part extending from its central part in the distal direction. The cylindrical part may be designed for connection with the connector.

The cylindrical part may comprise a circumferential groove and the connector may comprise a circumferential projection, said groove and said projection being adapted to be mutually engaged.

The coupling element may comprise at least two axially extending structures.

At least two of the axially extending structures may be disposed opposite to each other.

The first set of teeth of the dial sleeve may comprise radially-oriented teeth and the second set of teeth of the dial sleeve may comprise axially-oriented teeth.

The coupling element may comprise an external thread and the dial sleeve may comprise an internal longitudinal rib, the injection device being provided with a blocking ring comprising at least one projection on its internal surface for cooperation with the external thread of the coupling element and comprising a longitudinal groove for cooperation with the internal longitudinal rib of the dial sleeve.

The piston rod may have a non-circular cross-section and the driving sleeve may have a corresponding non-circular cross section so that the piston rod is rotationally coupled with the driving sleeve when it is placed inside the driving sleeve.

The ratchet wheel may comprise a ledge, the ledge being adapted to block the distal end of the driving sleeve in order to prevent it from moving axially in the distal direction.

The ratchet wheel may be fixed in the housing by means of a locking ring fixedly mounted in the housing.

The driving sleeve may comprise a flange and the locking ring may comprise a ledge, the flange abutting the ledge so that to prevent the driving sleeve from moving axially in the proximal direction.

The technical advantage of the delivery device as disclosed relies in that it allows for easy and precise dose setting due to the cooperation of the axially extending structures of the coupling element and the teeth of the dial sleeve. Furthermore, the presented device does not require any intermediary parts between the coupling element and the dial sleeve. The construction is robust, while the design of the elements as well as their limited number contribute to relatively easy manufacture and reduced costs.

Exemplary embodiments of the invention are presented in the appended drawings in which:

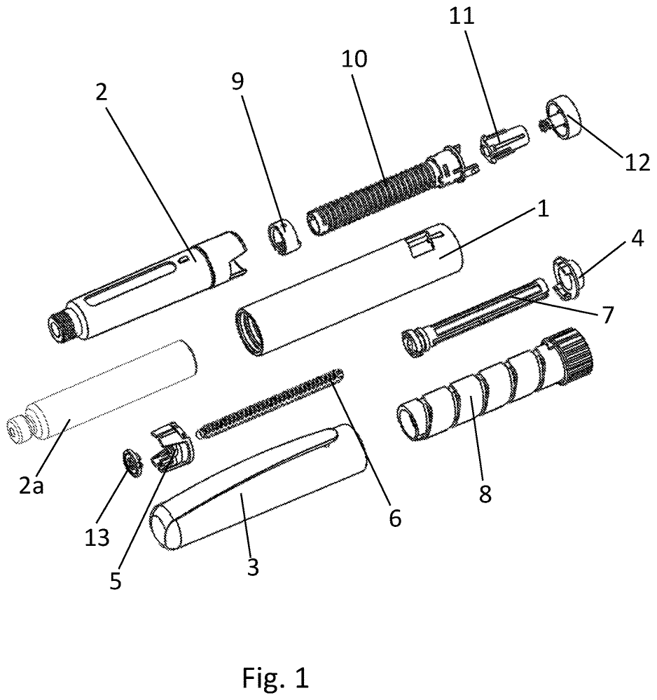

FIG. 1 shows an isometric and exploded view of the drug injection device according to a first embodiment;

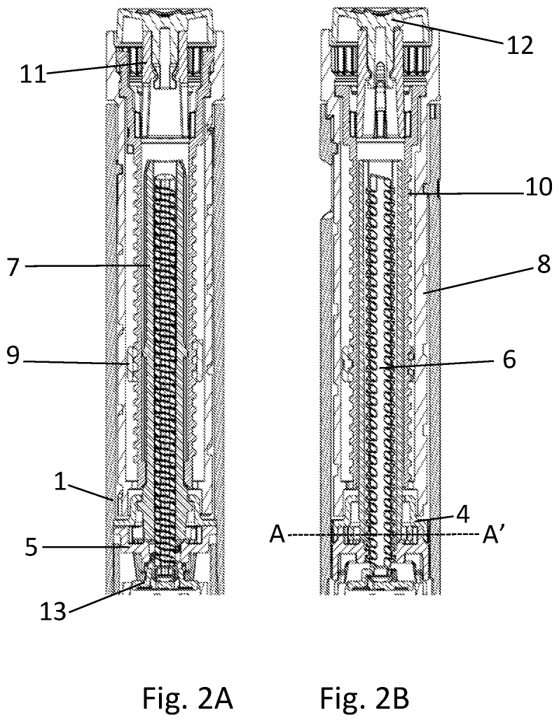

FIGS. 2A and 2B show two longitudinal sections of the drug injection device according to the first embodiment;

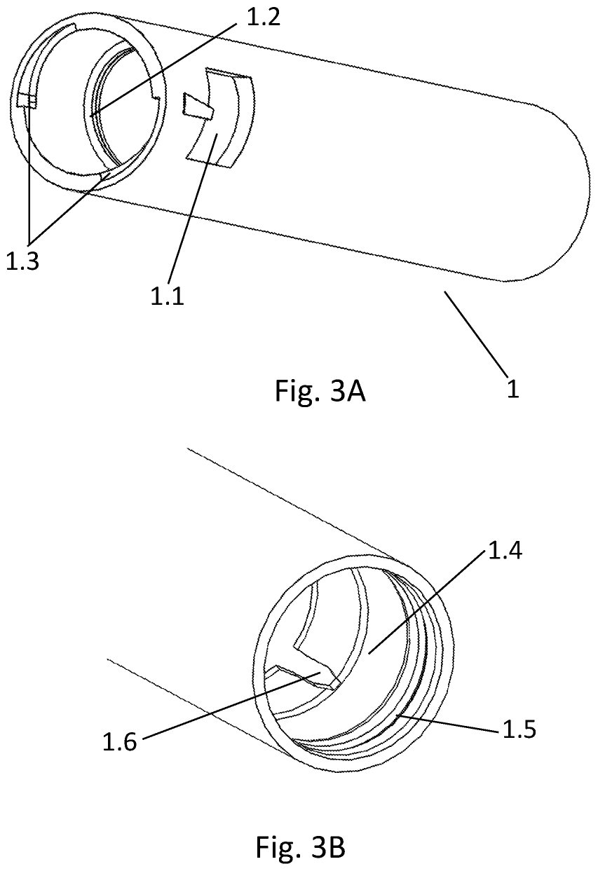

FIGS. 3A and 3B show two isometric views of a housing of the drug injection device according to the first embodiment;

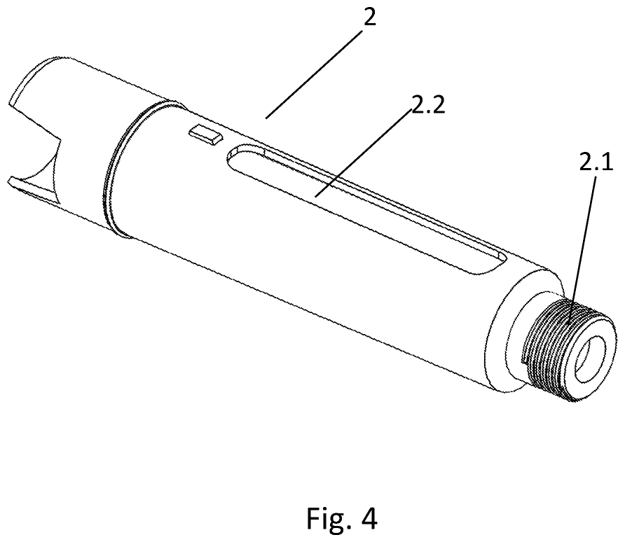

FIG. 4 shows an isometric view of a cartridge holder of the drug injection device according to the first embodiment;

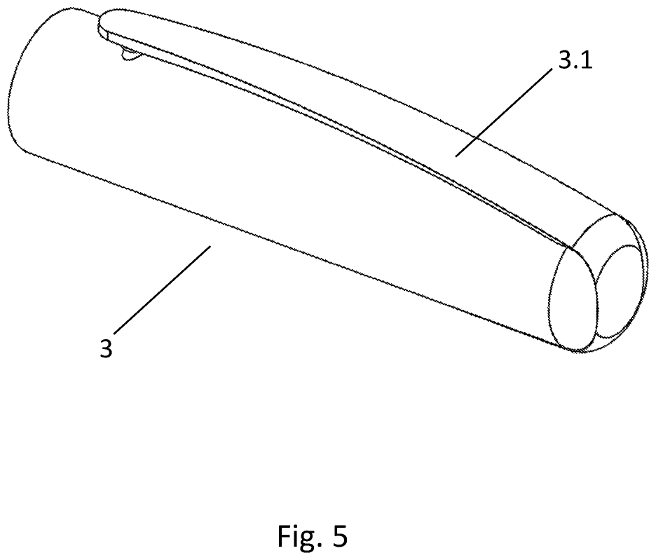

FIG. 5 shows an isometric view of a cap of the drug injection device according to the first embodiment;

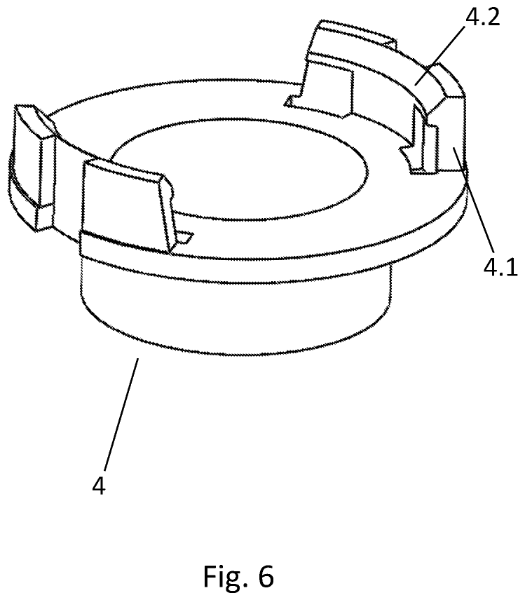

FIG. 6 shows an isometric view of a locking ring of the drug injection device according to the first embodiment;

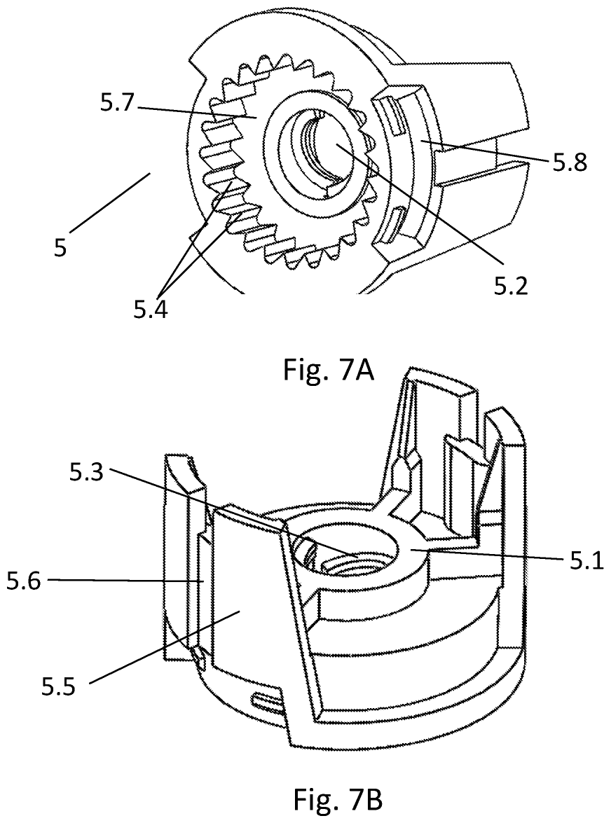

FIGS. 7A and 7B show two isometric views of a ratchet wheel of the drug injection device according to the first embodiment;

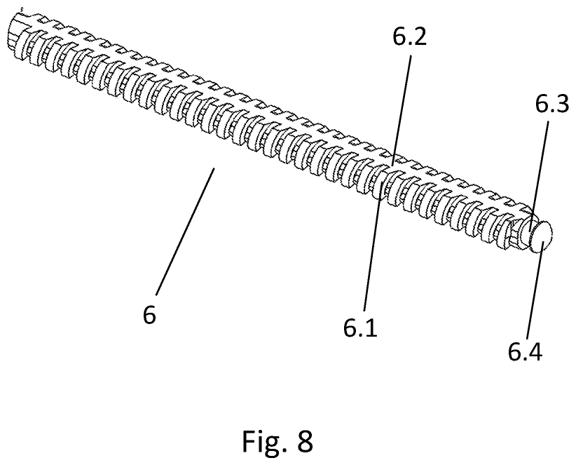

FIG. 8 shows an isometric view of a piston rod of the drug injection device according to the first embodiment;

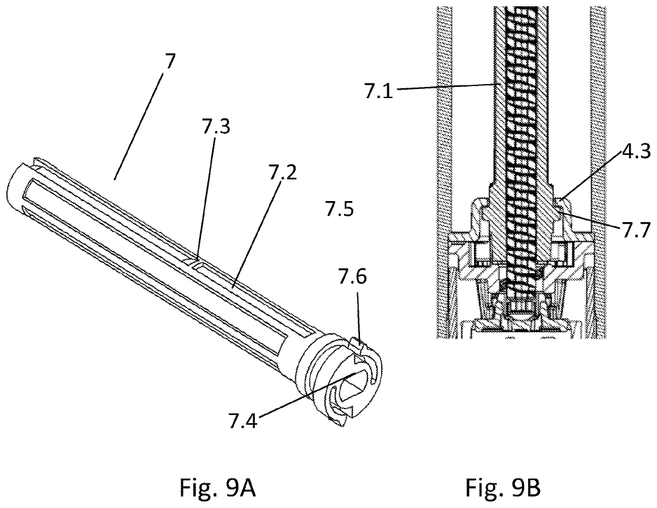

FIG. 9A shows an isometric view of a driving sleeve of the drug injection device according to the first embodiment;

FIG. 9B shows a partial longitudinal section of a driving sleeve of the drug injection device according to the first embodiment;

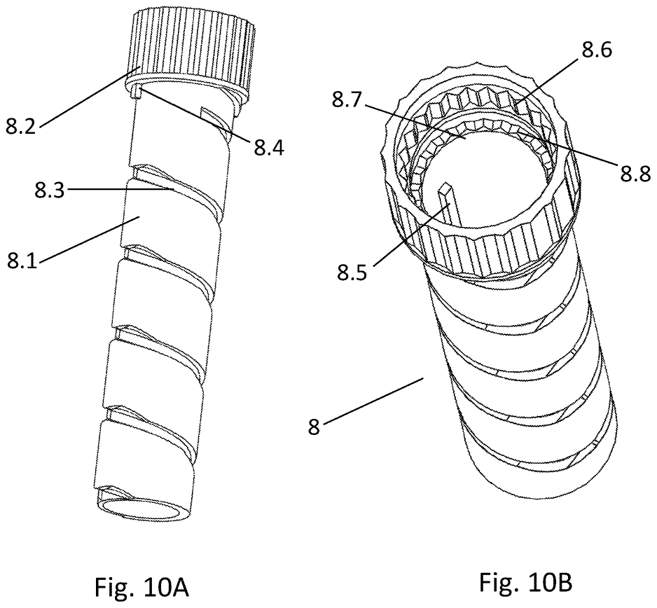

FIGS. 10A and 10B show two isometric views of a dial sleeve of the drug injection device according to the first embodiment;

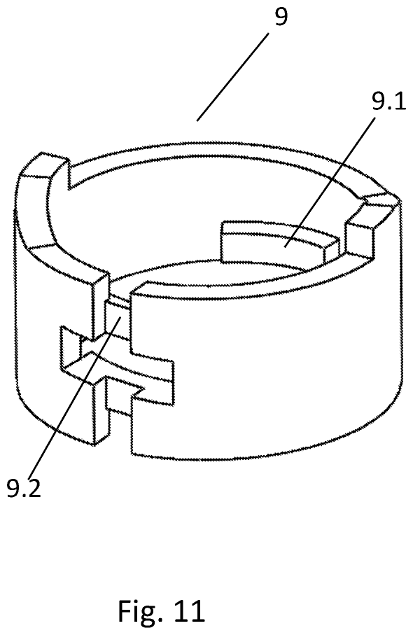

FIG. 11 shows an isometric view of a blocking ring of the drug injection device according to the first embodiment;

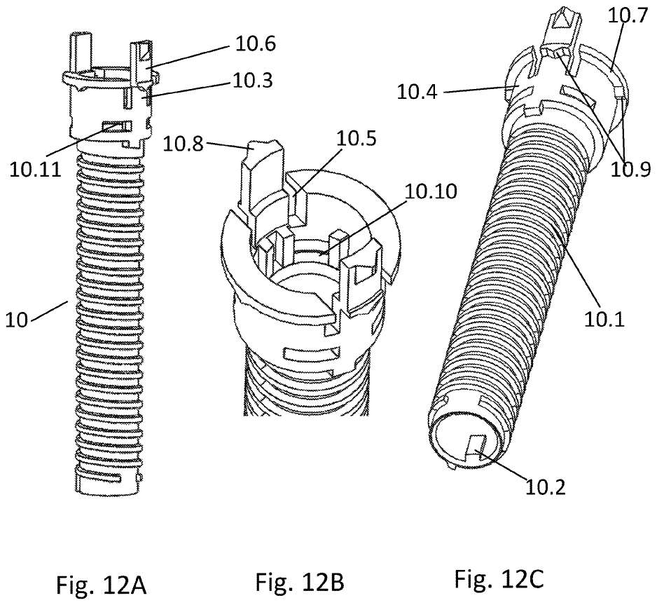

FIGS. 12A, 12B and 12C show three isometric views of a coupling element of the drug injection device according to the first embodiment;

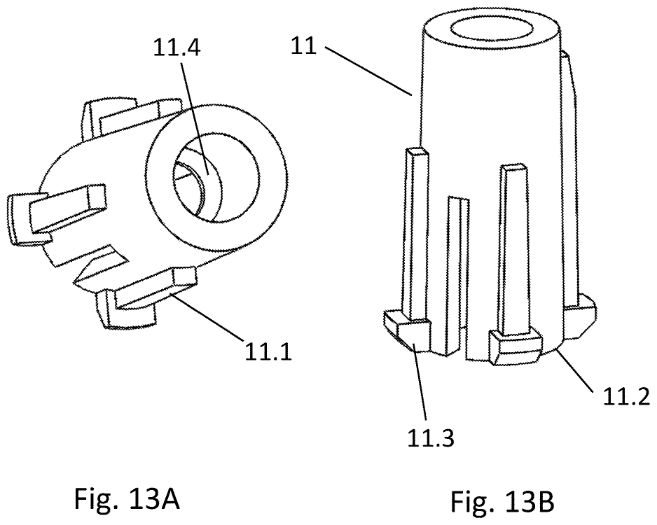

FIGS. 13A and 13B show two isometric views of a connector of the drug injection device according to the first embodiment;

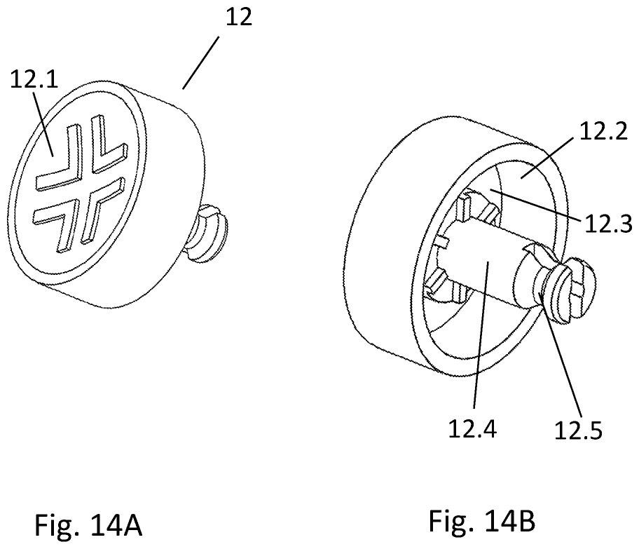

FIGS. 14A and 14B show two isometric views of a push button of the drug injection device according to the first embodiment;

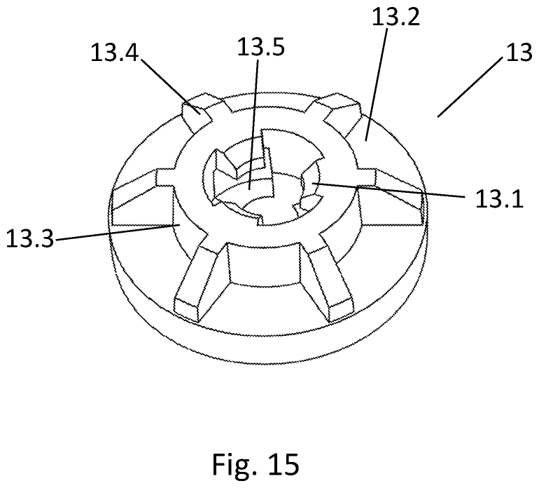

FIG. 15 shows an isometric view of a piston rod foot of the drug injection device according to the first embodiment;

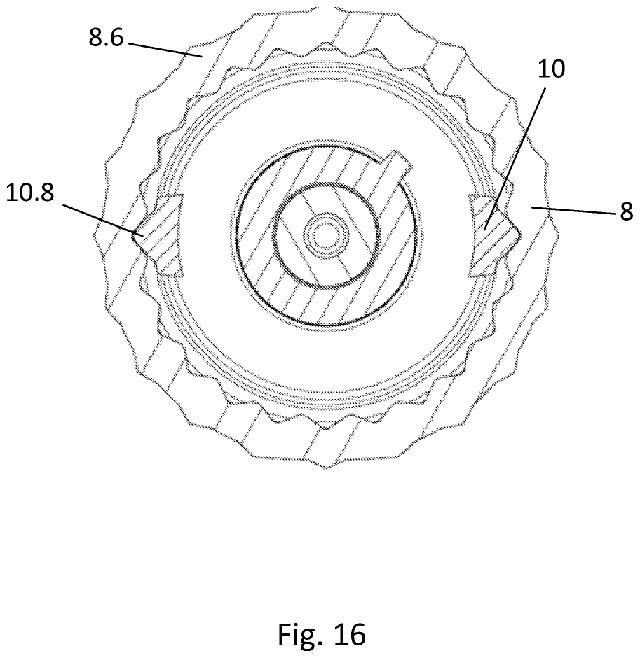

FIG. 16 presents a cross-section of a dial sleeve and a coupling element of the drug injection device according to the first embodiment, illustrating cooperation of these elements during dose setting and dose correcting;

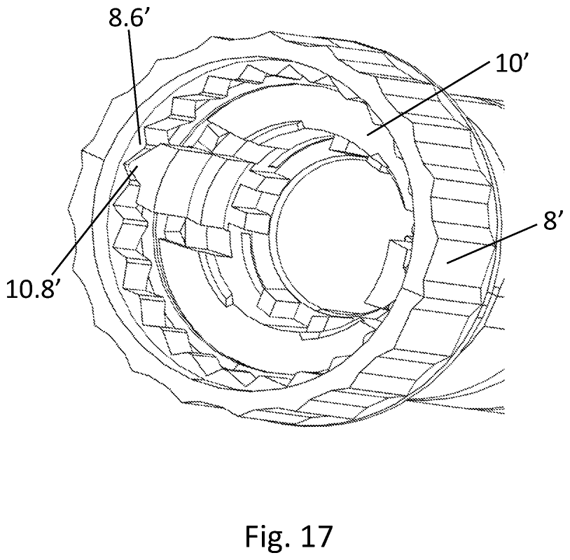

FIG. 17 presents an isometric and top view of a dial sleeve and a coupling element of the drug injection device according to any embodiment, illustrating an exemplary arrangement of the asymmetric teeth;

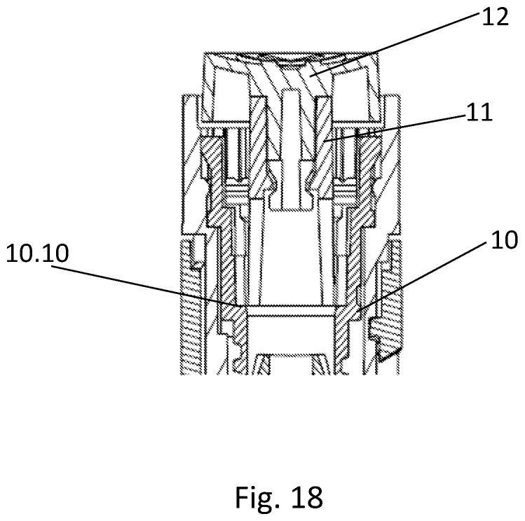

FIG. 18 presents a partial longitudinal section of a sub-assembly of the push button, the connector and the coupling element of the drug injection device according to the first embodiment;

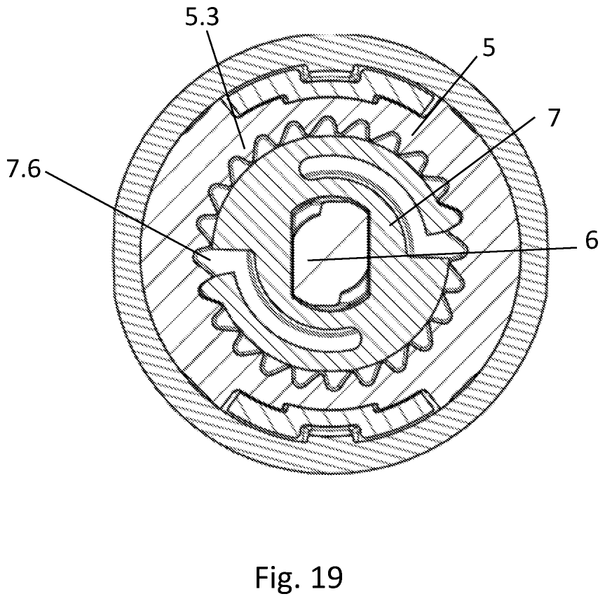

FIG. 19 presents a cross section taken along the line A-A′ indicated in FIG. 2B, illustrating cooperation of driving sleeve arms and ratchet wheel teeth;

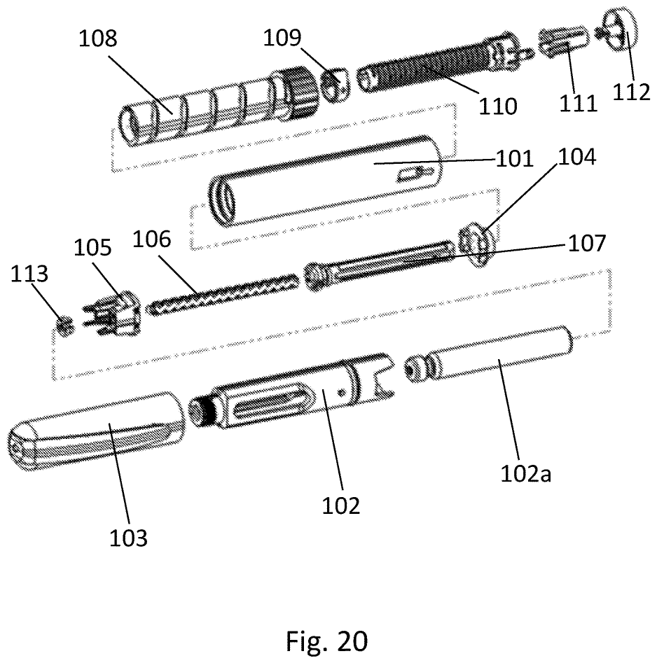

FIG. 20 shows an isometric and exploded view of the drug injection device according to a second embodiment

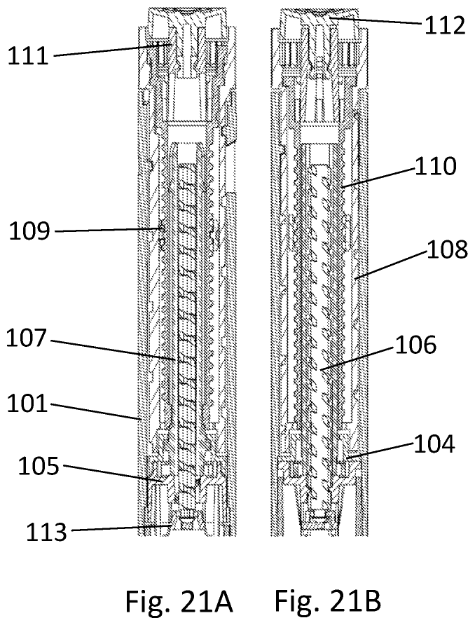

FIGS. 21A and 21B show the two longitudinal sections of the drug injection device according to the second embodiment;

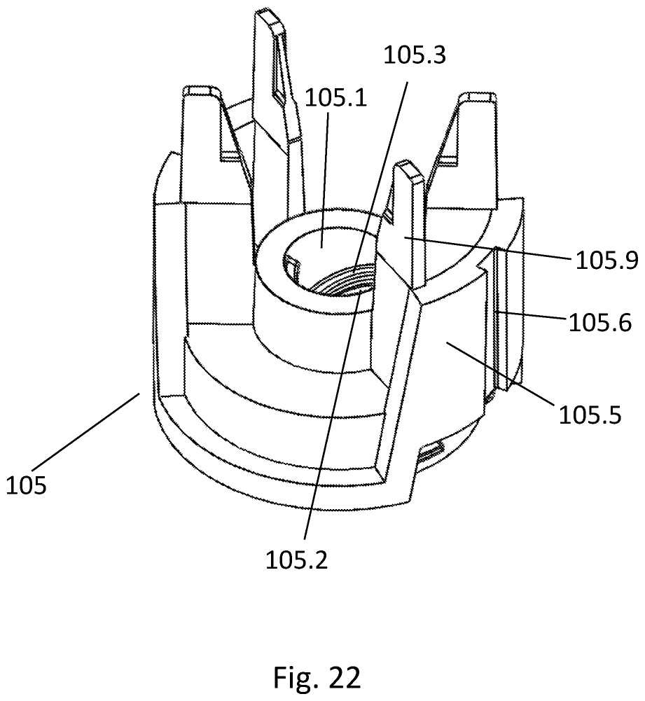

FIG. 22 shows an isometric view of a ratchet wheel of the drug injection device according to the second embodiment;

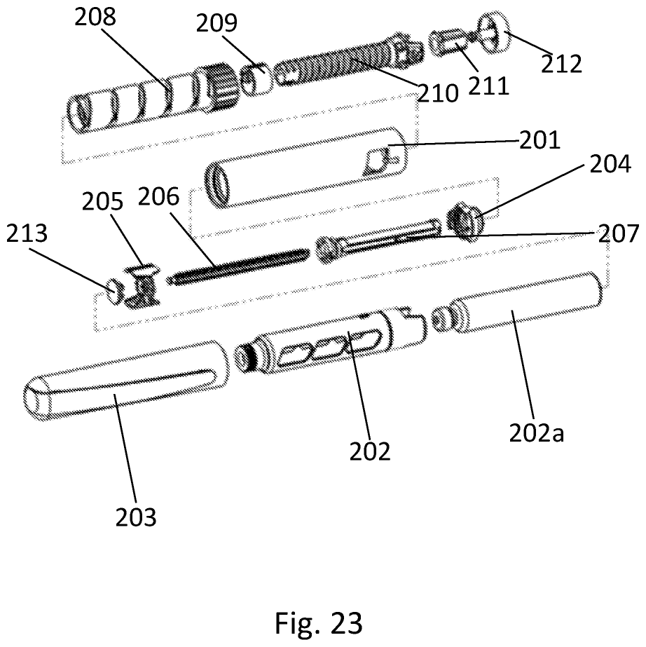

FIG. 23 shows an isometric exploded view of the drug injection device according to a third embodiment;

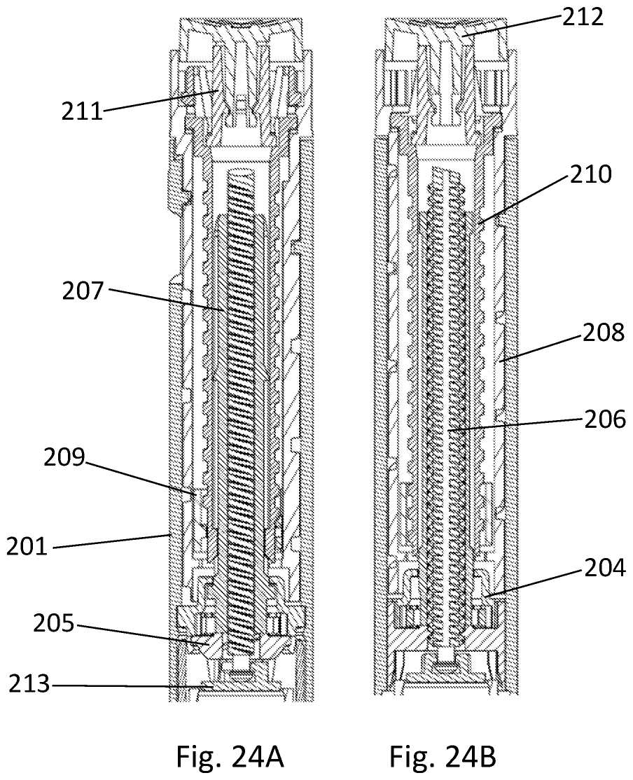

FIGS. 24A and 24B show two longitudinal sections of the drug injection device according to the third embodiment;

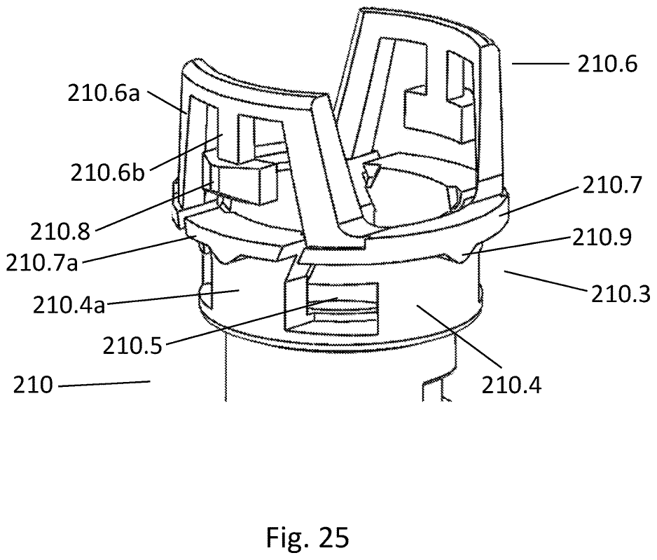

FIG. 25 shows a coupling element of the drug injection device according to the third embodiment

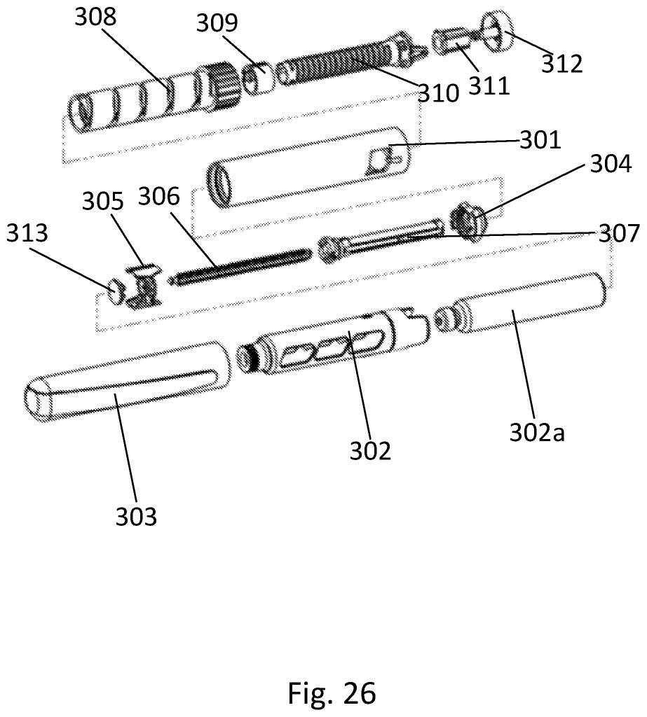

FIG. 26 shows an isometric and exploded view of the drug injection device according to a fourth embodiment

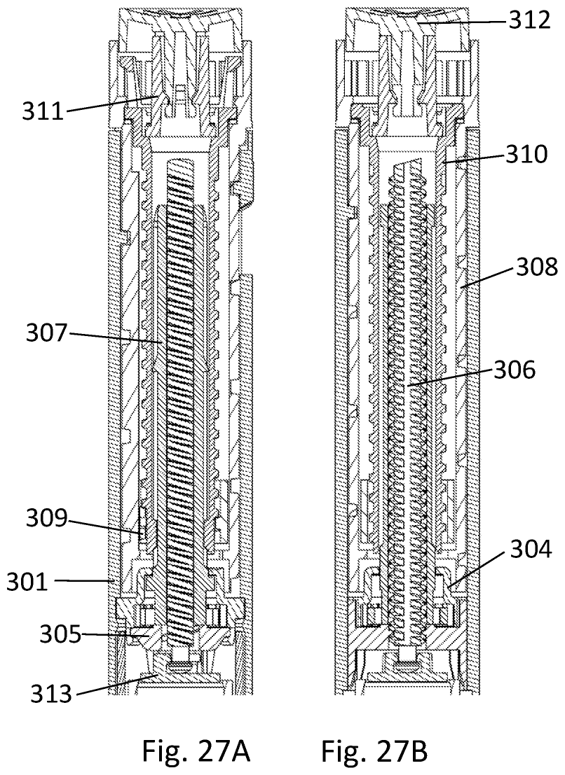

FIG. 27A,B show the cross-sections of the drug injection device according to the fourth embodiment

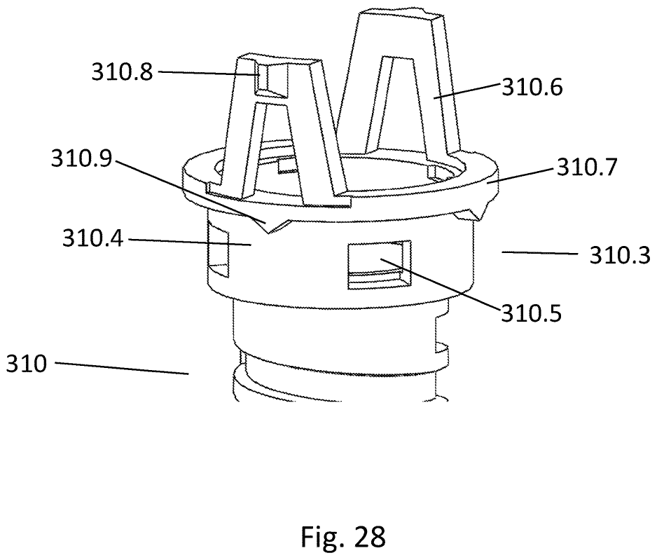

FIG. 28 shows a coupling element of the drug injection device according to the fourth embodiment

In the following description of the embodiments of the claimed device, and in the entire description, unless otherwise specified, the “distal direction” means the direction towards the dispensing end of the device, or the “distal end” i.e. the end to which the needle assembly is mounted before the injection. The “proximal direction” means the opposite direction i.e. the direction towards the rotatable element used for setting a dose to be injected, or the “proximal end”, for example a respective part of a dial sleeve.

The injection device as disclosed can be used with various cartridges containing a suitable liquid substance. In particular, a standard glass cartridge with a volume of 1.5 ml, 2.4 ml or 3 ml may be used. It may contain a drug, a vaccine or other ingredients to be injected.

The injection device as disclosed can be used for delivering a variety of drugs. The term “drug” or “medicament”, as used herein, means a pharmaceutical formulation containing at least one pharmaceutically active compound. These pharmaceutically active compounds can be used alone or in combination, in any pharmaceutically acceptable salt or solvate form and include without any limitation: compounds having a molecular weight up to 1500 Da such as for example zoledronate, peptides, proteins, polysaccharides such as glycosaminoglycans, hyaluronic acids, heparins, low molecular weight heparins or derivatives thereof, or sulphated, e.g. poly-sulphated forms of the above-mentioned polysaccharides, and/or a pharmaceutically acceptable salt thereof, vaccines, polynucleotides, oligonucleotides, deoxyribonucleic acids (DNAs), ribonucleic acids (RNAs), enzymes, antibodies or glycoproteins or fragments thereof such as denosumab, panitumumab, hormones such as hypophysis or hypothalamus hormones, for example gonadotropin or gonadotropin releasing hormone, desmopressin, gonadorelin, triptorelin, terlipressin, leuprorelin, buserelin, goserelin, nafarelin, lutropin, menotropin, follicle-stimulating hormone or their analogs, follitropin, parathyroid hormone, teriparatide, abaloparatide or other parathyroid hormone analogs, calcitonin, growth hormone, somatropin, insulin or insulin analogs or derivatives, glucagon-like peptides and their analogs or agonists, incretin mimetics, amylin or amylin agonist or analogs, gastric inhibitory peptides or analogs. Pharmaceutically acceptable salts are for example acid addition salts and basic salts. Acid addition salts can be HCl or HBr salts. Basic salts are for example salts having a cation selected from alkali or alkaline, e.g. Na+, or K+, or Ca2+, or an ammonium ion substituted or not. Pharmaceutically acceptable solvates are for example hydrates.

The drugs that may be used in the device according to the invention can be useful for the treatment and/or the prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis or can be used as nutritional formulations.

The drugs may be presented in a liquid form suitable for injection including solutions, fine suspensions, liquids obtained after reconstitution of a lyophilized solid or gels.

The embodiments described below can be combined or partially combined with each other with their respective features rearranged, added, omitted etc. within the scope of the present invention. In this way possible additional embodiments may be obtained as well.

The injection device according to a first embodiment of the invention is shown in FIGS. 1-18.

As shown in FIGS. 1, 2A and 2B the injection device according to the first embodiment comprises a driving assembly mounted in a housing 1. The housing 1 is connected with a cartridge holder 2, in which a cartridge 2a containing a drug can be held.

As shown specifically in FIGS. 3A and 3B, the housing 1 comprises a substantially cylindrical body with an inspection window 1.1 through which an indication of the volume of the currently set dose may be visible. A thread 1.2 is disposed on the internal surface of the housing 1. In the presented embodiment the thread 1.2 runs over less than a full revolution. Such a short thread allows for easier manufacturing of the housing by injection molding. Alternatively, the thread 1.2 may also extend spirally along the whole length of the housing 1 or it may have any suitable length in-between. In other embodiments, the thread 1.2 may be disposed on a separate element fixed in the housing 1. The housing 1 comprises on its internal surface a pair of recesses 1.3 at its proximal end. A circumferential notch 1.4 is disposed close to the distal end of the housing 1, the notch 1.4 running over the whole circumference of the housing 1. Distally from the notch 1.4, a rib 1.5 is located on the internal surface of the housing 1. The notch 1.4 and the rib 1.5 provide axial blocking for other parts of the injection device as described below. Additionally, two longitudinal ribs 1.6 provide rotational blocking.

The cartridge holder 2 shown in FIG. 4, is preferably fixedly attached to the housing 1. The cartridge holder 2 holds the cartridge 2a. At the distal end of the cartridge holder 2, a threaded part 2.1 is disposed, on which a needle assembly can be mounted by a user before injection. Alternatively, other means for mounting a specific needle may be used, like snap fit connection or key-groove connection.

After the assembly of the device, the cartridge holder 2 with the cartridge 2a disposed inside is being fixed to the housing 1, preferably in a manner preventing disengagement. The housing 1 and the cartridge holder 2 may be connected by means of a snap fit connection, a latch connection or any other means known to a skilled person. The housing 1 and the cartridge holder 2 may also be welded during the assembly by means of laser welding, ultrasonic welding or another method known to a skilled person. In other embodiments the cartridge holder 2 may be releasably connected to the housing 1 allowing exchanging the cartridge 2a.

The user can see the cartridge through the openings 2.2 in order e.g. to check how much liquid remains in the cartridge or to inspect drug consistence. Alternatively, the cartridge holder 2 may include transparent surfaces or the whole cartridge holder 2 may be made of a transparent material.

A cap 3, shown in FIG. 5, may be used for protecting and easier handling of the injection device. The cap 3 is preferably provided with a clip 3.1 allowing, for example, to attach the injection device to clothes or preventing its rolling. Advantageously, the length of the cap 3 can be adjusted so that it prevents putting the cap 3 on the device with the needle assembly mounted. This provides an additional safety means, especially when disposable needle assemblies are used.

The cap 3 can be releasably mounted to the cartridge holder 2, preferably by means of a snap connection.

The driving assembly mounted in the housing 1 comprises the following elements, the structure of which will be described below: a dial sleeve 8, a coupling element 10, a driving sleeve 7, a piston rod 6, a ratchet wheel 5, a locking ring 4, a push button 12, a connector 11 and a blocking ring 9.

The dial sleeve 8, shown in detail in FIGS. 10A-10B comprises a cylindrical body 8.1 with an enlarged portion 8.2 on its proximal end. The enlarged portion 8.2 has its outer diameter larger than the rest of the dial sleeve 8. A helical groove 8.3 is disposed on the outer surface of the cylindrical body 8.1 forming a thread portion. The enlarged portion 8.2 may comprise a structure on its outer surface for facilitating gripping by the user. In the presented embodiment said structure comprises vertical external ribs.

Two protrusions 8.4 are disposed on the dial sleeve 8, adjacent to the distal end of the enlarged portion 8.2. The protrusions 8.4 have their edges designed to define the initial position of the dial sleeve 8 in the housing, i.e. the position corresponding to the zero dose set, wherein said edges are substantially parallel to the longitudinal axis of the device. The zero dose position corresponds to the position in which the protrusions 8.4 abut the recesses 1.3 of the housing 1. Hence, when the dial sleeve 8 is rotated in the direction corresponding to the dose correcting or dose injecting, said rotation is stopped once the protrusions 8.4 engage the recesses 1.3.

A longitudinal rib 8.5 is disposed on the inner surface of the cylindrical body 8.1. The rib 8.5 provides linear guiding for the blocking ring 9 along the main longitudinal axis of the device. A first series of teeth 8.6 are disposed on the inner circumference of the enlarged portion 8.2. The teeth 8.6 extend radially. The dial sleeve 8 further comprises an inner flange 8.7 near its proximal end. A second series of teeth 8.8 extending axially are disposed on the flange 8.7. The number of the radial teeth 8.6 and the axial teeth 8.8 is preferably the same.

The dial sleeve 8 preferably includes a plurality of numerals disposed helically on its outer surface, the numerals corresponding to the currently set dose to be visible through the inspection window 1.1 in the housing. The dial sleeve 8 may also include other indicators for providing the user with the necessary information about the dose and conduct of the dose setting and correcting and dose injecting stages. In particular, the dial sleeve 8 may include symbols including letters, for example ml or mg, dashes, long lines or dots. These markings can be provided on the dial sleeve 8 by means of the laser printing, pad printing or other technique known for the skilled person.

The helical groove 8.3 on the external surface of the cylindrical body 8.1 is engaged with the internal thread 1.2 of the housing 1. Consequently, the dial sleeve 8 can rotate and move axially in both distal and proximal directions.

The coupling element 10 is shown in detail in FIGS. 12A-12C. The coupling element 10 is a generally tubular member with a thread 10.1 disposed on its outer surface and two projections 10.2 disposed on its inner surface. The coupling element 10 comprises an enlarged part 10.3 at its proximal end. The enlarged part 10.3 comprises a generally cylindrical wall 10.4 in which indentations 10.5 are formed. Axially extending structures 10.6 are provided in said indentations 10.5, wherein the structures 10.6 protrude from the distal edges of the indentations 10.5 in the proximal direction. The wall 10.4 has an outer flange 10.7 at its proximal end. In the presented embodiment there are two axially extending structures 10.6 disposed opposite to each other and two indentations 10.5. Alternatively, other numbers of the axially extending structures and the indentations may be used.

In other embodiments there may be three indentations 10.5 or four indentations 10.5 in which respectively three or four axially extending structures 10.6 are disposed. In yet other embodiments the number of the indentations 10.5 is different than the number of the structures 10.6, as the number of the axially extending structures 10.6 located in a single indentation 10.5 may be more than one and an indentation 10.5 may be formed so that it does not comprise axially extending structures 10.6.

There may be only one structure 10.6. Preferably, the coupling element 10 comprises at least two extending structures 10.6. Most preferably, the number of the extending structures 10.6 is a divisor of the number of the first teeth 8.6 of the dial sleeve. In the first embodiment, there are 24 teeth 8.6. Each axially extending structure 10.6 comprises at its distal end a protrusion 10.8 projecting radially outward. Preferably, each protrusion 10.8 is formed by two symmetrically inclined walls. In this embodiment, the first teeth 8.6 have also symmetrically inclined walls. This results in that the values of the torque required for setting and correcting the dose are the same. Also, the click sounds generated during dose setting and dose correcting are identical. In some applications it may be desired to provide the user with a different type of feedback in which the click sound and/or the torque values are different for dose setting and dose correcting. In order to fulfill these requirements, in other embodiments the protrusions 10.8′ of the coupling element 10′ may have asymmetric profiles, the radial teeth 8.6′ of the dial sleeve 8′ may have asymmetric profiles or both the protrusions 10.8′ and the radial teeth 8.6′ may have asymmetric profiles. Such an example is shown in FIG. 17.

The axially extending structures 10.6 are integral with the coupling element 10 and they may be deflected radially. The axially extending structures 10.6 constitute integral parts of the coupling element 10, however in the first embodiment, two sections may be distinguished in each structure 10.6. The proximal section is shifted radially in the outward direction in comparison with the distal section. The axially extending structures 10.6 further comprise outer coupling teeth 10.9 located at the bottom of said proximal section. The coupling teeth 10.9 are designed for releasable engagement with the second teeth 8.8 of the dial sleeve 8 upon axial displacement of the coupling element 10 in the distal direction.

The protrusions 10.8 of the axially extending structures 10.6 engage the first teeth 8.6 of the dial sleeve 8. When the dial sleeve 8 and the coupling element 10 are rotated with respect to each other, the protrusions 10.8 may pass over the first teeth 8.6 of the dial sleeve 8 to the subsequent positions. This movement is possible in both directions of the relative rotation of the coupling element 10 and the dial sleeve 8.

A circumferential shelf 10.10 extends radially inward from the inner surface of the enlarged part 10.3. The shelf 10.10 is designed for cooperation with the connector 11. During operation of the device, the connector 11 displaces the coupling element 10 by pushing on the shelf 10.10. The coupling element 10 also comprises openings 10.11 formed as through-holes in the wall 10.4 for engagement with the protrusions 11.3 of the connector 11 to be described in reference to FIGS. 13A and 13B. However, this connection between the coupling element 10 and the connector 11 may be realized also in another way known to a skilled person.

As shown in FIGS. 9A and 9B, the driving sleeve 7 has a tubular body 7.1. It comprises on its outer surface two external grooves 7.2 extending in axial direction along the tubular body 7.1 of the driving sleeve 7. The external grooves 7.2 of the driving sleeve 7 engage the projections 10.2 disposed on the inner surface of the coupling element 10. Due to this engagement the coupling element 10 can only move axially with respect to the driving sleeve 7. Accordingly, their rotation with respect to each other is prevented.

Walls 7.3 are disposed inside the grooves 7.2 in order to provide maximum dose limiter. The position of the walls 7.3 along the groove corresponds to the maximum volume of drug which can be set and delivered in a single injection. By changing said position, the maximum dose volume may be therefore adjusted allowing to use the device for different applications as the drugs may require various dose regimens. The cross section of the tubular body 7.1 is non-circular and in the presented embodiment the inner surface of the tubular body comprises two flattened portions 7.4 disposed opposite to each other. The driving sleeve 7 comprises a distal end 7.5 having two radially extending ratchet arms 7.6. Alternatively, a different number of ratchet arms 7.6 may be disposed at the distal end 7.5 of the driving sleeve 7.

Near its distal end 7.5, the driving sleeve 7 comprises on its outer surface a flange 7.7 disposed close to the ratchet arms 7.6. Said flange 7.7 cooperates with the locking ring 4 in order to block the movement of the driving sleeve 7 in the proximal direction.

The driving sleeve 7 is axially fixed in the housing 1 due to cooperation with the locking ring 4 and a ratchet wheel 5. It is described in more detail below in reference to FIGS. 7a and 7b.

The piston rod 6, as may be seen in FIG. 8, is an elongated member with an outer thread 6.1. The piston rod 6 has non-circular cross section allowing its rotational coupling with the driving sleeve 7. In this embodiment the outer surface of the piston rod 6 comprises two flattened surfaces 6.2 disposed opposite to each other. Alternatively, the piston rod 6 may have a different number of the flattened surfaces, in particular only one flattened surface may be present or another non-circular shape may be designed, for example in a form of the key groove connection. In the present embodiment, the distal portion of the piston rod 6 comprises a narrower portion 6.3 and a widened rounded part 6.4 at the end.

In the first embodiment, the piston rod 6 is rotationally coupled with the driving sleeve 7 by the flattened surfaces 6.2 cooperating with the flattened portions 7.4 of the driving sleeve 7. Therefore, rotation of the driving sleeve 7 causes rotation of the piston rod 6.

The piston rod 6 is provided at its distal end with a piston rod foot 13 comprising a generally planar surface for engaging a piston located in the cartridge 2a. Said planar surface may comprise openings outside its central area. The piston rod foot 13, shown in detail in FIG. 15, comprises three latches 13.1 disposed circumferentially, preferably at equal angular distances from each other. The piston rod foot 13 comprises a disc-shaped part 13.2 in contact with the piston. In the presented embodiment, at the proximal side of said disc-shaped part 13.2 a ring-shaped wall 13.3 is provided. In the presented embodiment, the piston rod foot 13 comprises additional ribs 13.4 disposed outside the wall 13.3 and directed radially. They impart improved stiffness to the piston rod foot and facilitate its manufacture by injection moulding. The piston rod foot 13 has openings 13.5 in the disc-shaped part 13.2 which are aligned with the latches 13.1.

The piston rod foot 13 is engaged with the piston rod 6 by means of the latches 13.1 which are coupled with the piston rod end part 6.4. When the piston rod 6 and the piston rod foot 13 are moved against each other axially, the latches 13.1 are deflected radially inward by the widened end part 6.4 of the piston rod 6. The widened part 6.4 passes through the latches 13.1 which engage the narrowed portion 6.3. In this position the latches 13.1 prevent disassembly of the piston rod 6 and the piston rod foot 13, because they cannot be deflected radially outward due to their shape. After its assembly, the piston rod foot 13 is rotatably connected with the piston rod 6. This allows to reduce the friction during dose delivery, because the piston rod 6 may freely rotate with respect to the piston rod foot 13 engaging the piston, which is usually made of rubber and is tightly fitted inside the cartridge. The distal end of the piston rod 6 abuts the proximal surface of the piston rod foot 13 so that the pushing force is transferred in the distal direction.

FIGS. 7A and 7B show the ratchet wheel 5. The ratchet wheel 5 has a generally annular part 5.1 comprising an opening 5.2 with a thread 5.3 formed on its inner surface. The thread 5.3 cooperates with the outer thread 6.1 of the piston rod 6. As a consequence, the piston rod 6 is displaced axially when it rotates.

At the proximal side the ratchet wheel 5 comprises a circumferential end part on the inner surface of which a series of teeth 5.4 is disposed. The teeth 5.4 are oriented radially inward. Preferably, each tooth 5.4 has two sides with different slopes.

The ratchet wheel 5 is fixedly mounted in the housing 1 and it does not move during operation of the device. The teeth 5.4 of the ratchet wheel 5 cooperate with the ratchet arms 7.6 of the driving sleeve 7. The different slopes of the teeth 5.4 ensure unidirectional coupling of the ratchet arms 7.6 and the teeth 5.4. Consequently, the driving sleeve 7 may rotate only in the direction allowed by this unidirectional coupling. Rotation of the driving sleeve 7 in the opposite direction is prevented.

Further, the ratchet wheel 5 preferably comprises axially extending protrusions 5.5 on the outer surface of which longitudinal grooves 5.6 are formed for rotational fixing of the ratchet wheel 5 within the housing 1 through engagement of the grooves 5.6 with the longitudinal ribs 1.6 on the inner surface of the housing 1.

The movement of the driving sleeve 7 in distal direction is prevented by a ratchet wheel ledge 5.7 axially blocking the distal part 7.5 of the driving sleeve 7.

The locking ring 4, as shown in FIG. 6, is a generally ring-shaped element configured to be engaged with the ratchet wheel 5. For this purpose, the locking ring 4 is provided with axially extending protrusions 4.1 having hooks 4.2 at their ends and the ratchet wheel 5 comprises a corresponding number of outer recesses 5.8 for snap-fit engagement with the hooks 4.2.

The locking ring 4 is fixedly mounted in the housing 1 and it does not move during operation of the device. The proximal movement of the driving sleeve 7 is prevented by the flange 7.7 of the driving sleeve 7 abutting a ledge 4.3 of the locking ring 4.

In the presented embodiment, the push button 12, shown in detail in FIGS. 14A and 14B, is disposed at the proximal end of the injection device. It comprises an outer surface 12.1 to be pressed by the user for injection of a dose of medicament. The outer surface 12.1 may comprise tactile elements, for instance bulges, for increasing friction between the push button 12 and the user's finger. Symbols providing information about the drug contained in the cartridge may also be placed on the outer surface 12.1. Further, the push button 12 comprises a circumferential flange 12.2 extending axially from the outer surface 12.1 in the distal direction. The flange 12.2 facilitates precise positioning of the push button 12 in the dial sleeve 7. The push button 12 has also an inner surface 12.3 comprising a central area from which a cylindrical part in the form of a sleeve 12.4 protrudes distally. A circumferential groove 12.5 is formed on the outer surface of the cylindrical part 12.4 close to its distal end.

The push button 12 is connected with the coupling element 10 by means of a connector 11. The connector 11 is a generally cylindrical hollow element.

In the presented embodiment the connector 11, shown in FIGS. 13A and 13B, comprises axially extending ribs 11.1 disposed on the outer surface of the cylindrical element. The connector 11 has a ledge 11.2 at its distal end with outwardly directed protrusions 11.3, preferably aligned with the ribs 11.1. The connector 11 comprises a circumferential projection 11.4 disposed on the inner surface of the connector 11, close to the middle of its longitudinal dimension.

The push button 12 is axially fixed to the connector 11 by means of the groove 12.5 disposed on the cylindrical part 12.4 of the push button 12 engaged with the circumferential projection 11.4 of the connector 11. The protrusions 11.3 of the connector 11 are engaged with the indentations 10.5 of the coupling element 10 causing the connector 11 to be rotationally coupled with the coupling element 10. Alternatively the connector may be rotationally coupled with the push button and the coupling element may rotate with respect to both these elements. The connector 11 may transfer force axially to the coupling element 10 by means of its distal edge abutting a shelf 10.10 located inside the enlarged part 10.3 of the coupling element 10.

FIG. 11 shows the blocking ring 9. The blocking ring 9 is designed to prevent setting of a dose larger than the remaining volume of the medication in the cartridge.

The blocking ring 9 is a ring-shaped element comprising at least one projection 9.1 on its inner surface. In the presented embodiment, the blocking sleeve 9 comprises two projections 9.1. The projections 9.1 on the inner surface of the blocking ring 9 engage with the external thread 10.1 of the coupling element 10.

On the outer surface of the blocking ring 9 there is a gap 9.2 forming a longitudinal groove which cooperates with the longitudinal rib 8.5 of the dial sleeve 8. By means of this arrangement, the blocking ring 9 is rotationally coupled with the dial sleeve 8, while being rotatable with respect to the coupling element 10 due to the engagement of the thread 10.1 with the projections 9.1. Therefore, rotation of the blocking ring 9 with respect to the coupling element 10 causes axial displacement of the blocking ring 9. Alternatively, the blocking ring 9 might be rotationally coupled with the coupling element 10 and threadedly engaged with the dial sleeve 8, which would provide the same effect of axial displacement of the blocking ring 9 upon rotation of the dial sleeve 8 with respect to the coupling element 10.

Operation of the injection device according to the described embodiment of the invention is described in the following.

In order to set a dose, the user grasps and rotates the enlarged portion 8.2 of the dial sleeve 8 in one rotational direction, preferably clockwise. The dial sleeve 8 is rotated and simultaneously translated axially in the proximal direction due to its threaded engagement with the housing 1. During dose setting, the protrusions 10.8 of the coupling element 10 pass over the first teeth 8.6 of the dial sleeve 8 and move to the subsequent positions. As can be seen in FIG. 16, the sloped surfaces of the radial teeth 8.6 slide on a sloped surfaces of the protrusions 10.8 of the coupling element 10. The axial structures 10.6 of the coupling element 10 may be deflected radially to allow rotation of the dial sleeve 8. The rotation by an angle corresponding to the angular distance between two subsequent teeth 8.6 moves each protrusion 10.8 to its next position. Preferably, the rotation by one tooth 8.6 corresponds to an increase of the set dose by a single unit. The rotation produces an audible signal of a clicking sound, indicating to the user that the dose is being set.

During dose setting, the coupling element 10 is displaced axially in the proximal direction along with the dial sleeve 8 due to the flange 10.7 of the coupling element 10 resting on the flange 8.7 of the dial sleeve 8. The rotation of the dial sleeve 8 is not transferred to the coupling element 10 because the teeth 8.8 of the dial sleeve 8 are not coupled with the teeth 10.9 of the coupling element 10 and the teeth 8.8 can slide over the teeth 10.9 without engaging them. The coupling element 10 is splined to the driving sleeve 7 by means of the grooves 7.2 and the projections 10.2. During dose setting, the driving sleeve 7 is prevented from rotation by the engagement of its ratchet arms 7.6 with the teeth 5.3 of the ratchet wheel 5. Hence, during dose setting the coupling element 10 does not rotate and it is only displaced axially.

The maximum settable dose is determined by the blocking members in a form of the proximal surface of the projections 10.2 of the coupling element and the surface of the end walls 7.3. When a currently set dose is equal to the maximum dose settable, said surfaces abut each other and further axial movement of the coupling element 10 is blocked, because the driving sleeve 7 is fixed axially. As the coupling element 10 cannot move axially, the dial sleeve 8 cannot be further rotated and a larger dose cannot be set.

As the driving sleeve 7 does not rotate, the piston rod 6 and the coupling element 10 do not rotate either.

The rotation of the dial sleeve 8 causes the subsequent numerals disposed on the outer surface of the dial sleeve 8 to appear in the inspection window 1.1, so that they can be seen by the user. Also, the number of clicks which can be heard provides another information about the set dose. This may be particularly useful for people with poor eyesight. Additional elements may be provided for facilitating the reading of the dose value, as for example a magnifying glass disposed in the inspection window 1.1. Alternatively, an electronic module equipped with an appropriate sensor may measure the displacement of the driving assembly parts and calculate therefrom the currently set dose. Its value may be displayed to the user, for example by means of a LCD screen.

In order to reduce the currently set dose, the dial sleeve 8 can be rotated in the second, opposite direction. The dose setting and dose correcting directions may be freely chosen during manufacturing of the device. Depending on the direction of the thread between the dial sleeve 8 and the housing 1, the dose can be increased by clockwise or counterclockwise rotation of the dial sleeve 8 and reduced by counterclockwise or clockwise rotation, respectively.

During the dose correcting, the driving assembly operates inversely to its operation during the dose setting. The protrusions 10.8 of the coupling element 10 jump back into their previous positions in the ring of the first teeth 8.6 of the dial sleeve 8. Accordingly, the respective numerals corresponding to a lower dose appear in the inspection window 1.1.

During the dose correcting, the coupling element 10 moves axially but it does not rotate. The second teeth 8.8 of the dial sleeve 8 and the coupling teeth 10.9 of the coupling element 10 are not coupled and the torque generated by the rotating dial sleeve 8 is therefore not transferred to the coupling element 10.

When the set dose is to be injected, the push button 12 is pressed in the distal direction causing the connector 11 to push the coupling element 10. These cooperating elements are shown in the cross-section in FIG. 18. In the presented embodiment the connector 11 abuts the shelf 10.10. The coupling teeth 10.9 come to engagement with the teeth 8.8 of the dial sleeve 8. In this way, the coupling element 10 becomes rotationally coupled to the dial sleeve 8. Therefore, a force in the distal direction is exerted on the dial sleeve 8 by the push button 12 via the connector 11 and the coupling element 10. Consequently, the dial sleeve 8 moves axially in the distal direction while simultaneously rotating due to its threaded engagement with the housing 1. As the teeth 10.9 and 8.8 are mutually coupled, the coupling element 10 rotates with the dial sleeve 8. The driving sleeve 7 rotates because it is splined to the coupling element 10. The piston rod 6 also rotates due to the engagement of its flattened surfaces 6.2 with the flattened portions 7.4 of the driving sleeve 7. The rotating piston rod 6 is also translated axially due to the engagement of the outer thread 6.1 of the piston rod 6 with the thread 5.3 of the opening 5.2 in the ratchet wheel 5. During the rotation of the driving sleeve 7, the ratchet arms 7.6 jump over the teeth 5.3 of the ratchet wheel 5, presented in FIG. 19, providing an audible feedback that a dose is being injected.

The dose is injected until the protrusions 8.4 of the dial sleeve abut the recesses 1.3 of the housing 1, causing the further rotation of the dial sleeve 8 to be blocked and the dial sleeve 8 is back in its initial position. It is ready for a next dose to be set.

The injection device according to the first embodiment comprises a last dose limiting system, which prevents setting a dose exceeding the amount of the medicament remaining in the cartridge. The last dose limiting system comprises the blocking ring 9 cooperating with the dial sleeve 8 and the coupling element 10.

During the dose setting, the blocking ring 9 is rotated along with the dial sleeve 8 and it also moves axially in the proximal direction due to its threaded engagement with the coupling element 10. The longitudinal position of the blocking ring 9 along the thread 10.1 corresponds to the sum of the already injected doses plus the currently set dose. The blocking ring 9 is in its final position on the thread 10.1 when the at least one projection 9.1 of the blocking ring 9 abuts the end of the thread 10.1. Said final position corresponds to the situation where the currently set dose is equal to the amount of the medicament remaining in the cartridge 2a. In the final position, the rotation of the blocking ring 9 is prevented by the abutment of the projection 9.1 with the end of the thread 10.1 and the dial sleeve 8 cannot be rotated in the direction for increasing the dose because it is rotationally fixed with the blocking ring 9. In this way a dose larger than the amount of the medicament remaining in the cartridge may not be set.

Alternatively, other or additional blocking elements may be provided on the blocking ring 9 and at least one of the dial sleeve 8 and the coupling element 10. Said blocking elements may have a form of surfaces protruding from the blocking ring 9 and the dial sleeve 8 and/or the coupling element 10, the respective protruding surfaces abutting each other in the final position of the blocking ring 9.

The injection device according to a second embodiment is shown in FIGS. 20-22.

Most of the features of the device according to the second embodiment are analogous to those of the first embodiment and will not be described in detail.

Specifically, as shown in FIGS. 20, 21A, 21B, the injection device according to the second embodiment comprises a driving assembly mounted in a housing 101. The housing 101 is connected with a cartridge holder 102, in which a cartridge 102a containing a medicament can be held. Further, the driving assembly of this embodiment is mounted in the housing 101 and comprises the following elements: a dial sleeve 108, a coupling element 110, a driving sleeve 107, a threaded piston rod 106, a ratchet wheel 105, a locking ring 104, a push button 112, a connector 111 and a blocking ring 109.

The injection device according to the second embodiment differs from the above described first embodiment in that its ratchet wheel 105 has a different structure as described below.

As shown in FIG. 22, the ratchet wheel 105 of the second embodiment has a generally annular part 105.1 and a central cylindrical part in which the opening 105.2 is formed extending in the distal direction. A thread 105.3 is formed on the inner surface of the opening 105.2. The thread 105.3 cooperates with an the thread of the piston rod 106 (similar to that shown in FIG. 8).

The ratchet wheel 105 of the second embodiment comprises, similarly as in the first embodiment, axially extending protrusions 105.5 with longitudinal grooves 105.6 for rotational fixing of the ratchet wheel 105 within the housing 101.

As may be seen in FIG. 22, wings 105.9 extending in the distal direction are disposed on the distal surface of the ratchet wheel body between the protrusions 105.5 and the central cylindrical part. In the presented second embodiment there are four wings 105.9, but any other number of wings can be foreseen, i.e. one or more wings. The wings 105.9 provide a better fixing of the cartridge 102a in the cartridge holder 102 by holding the cartridge 102a tightly. This reduces the wobbling of the cartridge during operation of the device. The shape of the wings 105.9 may be configured to match the external dimensions of a specific cartridge to be used with the injection device.

The injection device according to a third embodiment is shown in FIGS. 23-25.

Most of the features of the device according to the third embodiment are analogous to those of the first embodiment and will not be described in detail.

Specifically, as shown in FIGS. 23, 24A, 24B, the injection device according to the third embodiment comprises a driving assembly mounted in a housing 201. The housing 201 is connected with a cartridge holder 202, in which a cartridge 202a containing a medicament can be held. Further, the driving assembly of this embodiment is mounted in the housing 201 and comprises the following elements: a dial sleeve 208, a coupling element 210, a driving sleeve 207, a threaded piston rod 206, a ratchet wheel 205, a locking ring 204, a push button 212, a connector 211 and a blocking ring 209.

The injection device according to the third embodiment differs from the above described first embodiment in that the enlarged part 210.3 located at the proximal end of the coupling element 210 has a different structure as described below.

As shown in FIG. 25, the enlarged part 210.3 comprises a wall 210.4 in which indentations 210.5 and axially extending structures 210.6 are formed. Each structure 210.6 is composed of a trapezoid part 210.6a protruding from the wall 210.4 and a T-shaped part 210.6b extending in the distal direction from the upper edge of the structure 210.6. A ratchet protrusion 210.8 projects from said T-shaped part. The structure 210.6 allows the protrusion 210.8 to be deflected radially. Preferably, each protrusion 210.8 has is formed of two symmetrically inclined walls.

The protrusions 210.8 engage the first teeth 208.6 of the dial sleeve 208. The teeth 208.6 extend radially. When the dial sleeve 208 and the coupling element 210 are rotated with respect to each other, the protrusions 210.8 may pass over the first teeth 208.6 of the dial sleeve 208 to the subsequent positions. This movement is possible in both directions.

The coupling teeth 210.9 are designed for releasable engagement with the second teeth 208.8 of the dial sleeve 208 upon axial displacement the coupling element 210 in the distal direction. The teeth 208.8 extend axially. In this third embodiment the coupling teeth 210.9 may be disposed on a flange 210.7 or on a flange 210.7a disposed on the additional protrusions 210.4a located in the indentations 210.5. Preferably, the coupling teeth 210.9 are disposed on both flanges 210.7 and 210.7a

The injection device according to a fourth embodiment is shown in FIGS. 26-28.

Most of the features of the device according to the fourth embodiment are analogous to those of the first embodiment and will not be described in detail.

Specifically, as shown in FIGS. 26, 27A, 27B, the injection device according to the fourth embodiment comprises a driving assembly mounted in a housing 301. The housing 301 is connected with a cartridge holder 302, in which a cartridge 302a containing a medicament can be held. Further, the driving assembly of this embodiment is mounted in the housing 301 and comprises the following elements: a dial sleeve 308, a coupling element 310, a driving sleeve 307, a threaded piston rod 306, a ratchet wheel 305, a locking ring 304, a push button 312, a connector 311 and a blocking ring 309.

The injection device according to the fourth embodiment differs from the above described first embodiment in that the enlarged part 310.3 located at the proximal end of the coupling element 310 has a different structure as described below.

As shown in FIG. 28, the enlarged part 310.3 comprises a wall 310.4. At the proximal edge of the wall 310.4 is an outer flange 310.7 from which axially extending structures 310.6 protrude between through holes 310.5 (as opposed to the indentations 10.5 and 210.5). In the presented fourth embodiment there are two axially extending structures 310.6 disposed opposite to each other. Alternatively, there may be another number of the structures 310.6. There may be just one structure 310.6 and preferably, there are at least two structures, most preferably the number of the structures 310.6 is a divisor of the number of the first teeth 308.6 of the dial sleeve. The teeth 308.6 extend radially. The structures 310.6 are trapezoid shaped, each comprising a protrusion 310.8 projecting radially outward and disposed at the proximal edge of the arc. Preferably, each protrusion 310.8 is formed by two symmetrically inclined walls. The structures 310.6 are integral with the coupling element 310 and they may be deflected radially. The coupling teeth 310.9 are disposed on the outer surface of the coupling element 310, said coupling teeth 310.9 being located distally from the structures 310.6 and directed axially towards the distal direction. The coupling teeth 310.9 are designed for releasable engagement with the second teeth 308.8, which extend axially of the dial sleeve 308 upon axial displacement the coupling element 310 in the distal direction.

The protrusions 310.8 of the structures 310.6 engage the first teeth 308.6 of the dial sleeve 308. When the dial sleeve 308 and the coupling element 310 are rotated with respect to each other, the protrusions 310.8 may pass over the first teeth 308.6 of the dial sleeve 308 to the subsequent positions. This movement is possible in both directions.

### LIST OF ELEMENTS

1, 101, 201, 301-housing
1.1—inspection window
1.2—thread
1.3—recesses
1.4—notch
1.5—rib
1.6—longitudinal rib
2, 102, 202, 302—cartridge holder
2.1—threaded part
2.2—openings
2a, 102a—cartridge
3—cap
3.1—clip
4, 104, 204, 304—locking ring
4.1—protrusions
4.2—hooks
5, 105, 205, 305—ratchet wheel
5.1, 105.1—annular part
5.2, 105.2—opening
5.3, 105.3—thread
5.4, 105.4—radial teeth
5.5, 105.5—protrusions
5.6—grooves
5.7—ledge
5.8—recesses
105.9—wings
6, 106, 206, 306—piston rod
6.1—outer thread
6.2—flattened surfaces
6.3—narrower portion
6.4—widened round end
7, 107, 207, 307—driving sleeve
7.1—tubular body
7.2—grooves
7.3—end wall
7.4—flattened portions
7.5—distal end
7.6—ratchet arms
7.7—flange
8, 108, 208, 308—dial sleeve
8.1—cylindrical body
8.2—enlarged portion
8.3—helical groove
8.4—protrusions
8.5—rib
8.6, 208.6, 308.6—first teeth
8.7—flange
8.8, 208.8, 308.8—second teeth
9, 109, 209, 309—blocking ring
9.1—projections
9.2—groove
10, 110, 210, 310—coupling element
10.1, 210.1, 310.1—thread
10.2, 210.2, 310.2—projections
10.3, 210.3, 310.3—enlarged part
10.4, 210.4, 310.4—wall
10.5, 210.5—indentations
310.5—through—holes
10.6, 210.6, 310.6—structures
210.6a—trapezoid part
210.6b—t-shaped part
10.7, 210.7, 310.7—flange
10.8, 210.8, 310.8—protrusions
10.9. 210.9, 310.9—coupling teeth
10.10—shelf
10.11—openings
11, 111, 211, 311—connector
11.1—ribs
11.2—ledge
11.3—protrusions
11.4—circumferential projection
12, 112, 212, 312—push button
12.1—outer surface
12.2—flange
12.3—inner surface
12.4—cylindrical part
12.5—circumferential groove
13, 113, 213, 313—piston rod foot
13.1—latches
3.2—disc-shaped part 1
13.3—wall
13.4—ribs
13.5—openings

1. A drug injection device comprising
a housing comprising an internal thread,
a push button at its proximal end and, a driving assembly mounted in the housing, the driving assembly comprising:
an axially movable and rotatable dial sleeve (8) comprising an external thread cooperating with the internal thread of the housing,
an axially movable and rotatable coupling element within the dial sleeve,
an axially immovable and rotatable driving sleeve within the coupling element and rotationally coupled therewith, the driving sleeve comprising at least one radially extending ratchet arm at its distal end,
an axially movable and rotatable piston rod comprising an external thread, the piston rod being rotationally coupled with the driving sleeve,
a ratchet wheel fixed in the housing and comprising a series of radial teeth cooperating with said at least one ratchet arm for blocking rotation of the driving sleeve in one direction and allowing rotation of the driving sleeve in the opposite direction, the ratchet wheel further comprising an opening having an internal thread cooperating with the external thread of the piston rod,
wherein the dial sleeve comprises a proximal enlarged portion comprising a first set of teeth and a second set of teeth located distally from the first teeth,
wherein the coupling element comprises at least one axially extending structure, the structure comprising a protrusion engaging the first set of teeth of the enlarged portion, the protrusion passing over first set of teeth into subsequent positions during dose setting and dose correcting,
wherein the coupling element comprises the coupling teeth, and
wherein the coupling element being axially movable between a dose setting/correcting position in which the coupling teeth are decoupled from said second set of teeth, and a dose delivery position in which the coupling teeth are coupled with said second set of teeth.

2. The drug injection device according to claim 1, wherein the coupling element is axially movable between the dose setting/correcting position and the dose delivery position by means of the push button.

3. The drug injection device according to claim 1, wherein the coupling element is engaged with the push button by intermediary of a connector.

4. The drug injection device according to claim 3, wherein the coupling element and the connector may rotate with respect to the push button.

5. The drug injection device according to claim 3, wherein the connector comprises a ledge and the coupling element comprises an internal shelf, the ledge being adapted to abut the internal shelf.

6. The drug injection device according to claim 5, wherein the connector comprises at least two protrusions on its ledge and the coupling element comprises openings on its enlarged part wall, the at least two protrusions being designed for engagement with said openings.

7. The drug injection device according to claim 1, wherein the push button comprises a cylindrical part extending from its central part in the distal direction, the cylindrical part being designed for connection with the connector.

8. The drug injection device according to claim 6, wherein the cylindrical part comprises a circumferential groove and the connector comprises a circumferential projection, the groove and the projection being adapted to be engaged.

9. The drug injection device according to claim 1, wherein the coupling element comprises at least two axially extending structures.

10. The drug injection device according to claim 8, wherein at least two of the axially extending structures are disposed opposite to each other.

11. The drug injection device according to claim 1, wherein the first set of teeth comprises radially-oriented teeth and the second set of teeth comprises axially-oriented teeth.

12. The drug injection device according to claim 1, wherein the coupling element comprises an external thread and the dial sleeve comprises an internal longitudinal rib, the injection device being provided with a blocking ring comprising at least one projection on its internal surface for cooperation with the external thread of the coupling element and comprising a longitudinal groove for cooperation with the internal longitudinal rib of the dial sleeve.

13. The drug injection device according to claim 1, wherein the piston rod has a non-circular cross section and the driving sleeve has a corresponding non-circular cross section so that the piston rod is rotationally coupled with the driving sleeve when it is placed inside the driving sleeve.

14. The drug injection device according to claim 1, wherein the ratchet wheel comprises a ledge and the driving sleeve comprises a distal end, wherein the ledge is adapted to block the distal end and to prevent the driving sleeve from moving axially in the distal direction.

15. The drug injection device according to claim 1, wherein the ratchet wheel is fixed in the housing by a locking ring fixedly mounted in the housing.

16. The drug injection device according to claim 14, wherein the driving sleeve comprises a flange and the locking ring comprises a ledge, wherein the flange abuts the ledge so that the driving sleeve is prevented from moving axially in the proximal direction.
